(19) United States
(12) Patent Application Publication
(10) Pub. No.: US 2026/0327262 A1
(43) Pub. Date: Oct. 22, 2026

(54) BICYCLIC PEPTIDE LIGANDS SPECIFIC FOR EPHA2

(71) Applicant: BicycleTx Limited, Cambridge (GB)

(72) Inventors: Punit Upadhyaya, Cambridge (GB); Johanna Lahdenranta, Cambridge (GB); Gemma Elizabeth Mudd, Cambridge (GB); Kevin McDonnell, Cambridge (GB)

(73) Assignee: BicycleTx Limited, Cambridge (GB)

(21) Appl. No.: 19/464,790

(22) Filed: Jan. 30, 2026

Related U.S. Application Data
(63) Continuation of application No. 17/630,314, filed on Jul. 30, 2020, now Pat. No. 12,570,695.
(60) Provisional application No. 62/880,191, filed on Jul. 30, 2019.
(60) Provisional application No. 62/910,088, filed on Oct. 3, 2019.
(60) Provisional application No. 62/931,442, filed on Nov. 6, 2019.
(60) Provisional application No. 63/022,667, filed on May 11, 2020.
(60) Provisional application No. 63/024,715, filed on May 14, 2020.

(51) Int. Cl. C07K 7/08; A61K 9/00; A61K 38/00; A61K 38/12; A61K 47/64; A61P 35/00; C07K 11/02; C07K 14/00; C07K 14/705

(52) U.S. Cl. CPC C07K 7/08; A61K 9/0019; A61K 38/12; A61K 47/641; A61P 35/00; C07K 11/02; C07K 14/001; C07K 14/70578; A61K 38/00

(57) ABSTRACT

The present invention relates to polypeptides which are covalently bound to non-aromatic molecular scaffolds such that two or more peptide loops are subtended between attachment points to the scaffold. In particular, the invention describes peptides which are high affinity binders of the Eph receptor tyrosine kinase A2 (EphA2). The invention also relates to pharmaceutical compositions comprising said peptide ligands and to the use of said peptide ligands in preventing, suppressing or treating a disease or disorder characterised by overexpression of EphA2 in diseased tissue (such as a tumour).

### RELATED APPLICATIONS

This application is a continuation of U.S. application Ser. No. 17/630,314, filed Jan. 26, 2022, which is a U.S. national stage application under 35 U.S.C. § 371 of International Application No. PCT/GB2020/051829, filed Jul. 30, 2020, which claims priority under 35 U.S.C. § 119 to U.S. Provisional Application No. 62/880,191, filed Jul. 30, 2019, U.S. Provisional Application No. 62/910,088, filed Oct. 3, 2019, U.S. Provisional Application No. 62/931,442, filed Nov. 6, 2019, U.S. Provisional Application No. 63/022,667, filed May 11, 2020, and U.S. Provisional Application No. 63/024,715, filed May 14, 2020. The contents of each of the foregoing related applications are incorporated herein by reference in their entirety.

### REFERENCE TO AN ELECTRONIC SEQUENCE LISTING

The contents of the electronic sequence listing (B170170071US01-SEQ-HJD.xml; Size: 172,047 bytes; and Date of Creation: Dec. 3, 2025) are herein incorporated by reference in their entirety.

### FIELD OF THE INVENTION

The present invention relates to polypeptides which are covalently bound to non-aromatic molecular scaffolds such that two or more peptide loops are subtended between attachment points to the scaffold. In particular, the invention describes peptides which are high affinity binders of the Eph receptor tyrosine kinase A2 (EphA2). The invention also relates to pharmaceutical compositions comprising said peptide ligands and to the use of said peptide ligands in preventing, suppressing or treating a disease or disorder characterised by overexpression of EphA2 in diseased tissue (such as a tumour).

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule (tris-(bromomethyl)benzene).

### SUMMARY OF THE INVENTION

According to a first aspect of the invention, there is provided a peptide ligand specific for EphA2 comprising a polypeptide comprising at least three reactive groups, separated by at least two loop sequences, and a molecular scaffold, which is 1,1′,1″-(1,3,5-triazinane-1,3,5-triyl)triprop-2-en-1-one, which forms covalent bonds with the reactive groups of the polypeptide such that at least two polypeptide loops are formed on the molecular scaffold, wherein the peptide ligand comprises an amino acid sequence selected from:
A-[HArg]-D-Cᵢ[HyP]LVNPLCᵢᵢLHP[dD]W[HArg]Cᵢᵢᵢ (SEQ ID NO: 1; herein referred to as BCY9594);
[PYA]-A-[HArg]-D-Cᵢ[HyP]LVNPLCᵢᵢLHP[dD]W[HArg]Cᵢᵢᵢ (SEQ ID NO: 2; herein referred to as BCY11813);
Ac-A-[HArg]-D-Cᵢ[HyP]LVNPLCᵢᵢLHP[dD]W[HArg]Cᵢᵢᵢ)-[K(PYA)] (SEQ ID NO: 3; herein referred to as BCY11814);
Ac-A-[HArg]-D-Cᵢ[HyP]LVNPLCᵢᵢLHP[dD]W[HArg]Cᵢᵢᵢ-K (SEQ ID NO: 4; herein referred to as BCY12734);
[NMeAla]-[HArg]-D-Cᵢ[HyP]LVNPLCᵢᵢLHP[dD]W[HArg]Cᵢᵢᵢ (SEQ ID NO: 5; herein referred to as BCY13121);
[PYA]-[B-Ala]-[Sar₁₀]-VGP-CᵢLWDPTPCᵢᵢANLHL[HArg]Cᵢᵢᵢ (SEQ ID NO: 6; herein referred to as BCY8941);
Ac-A-[HArg]-D-Cᵢ[HyP]LVNPLCᵢᵢL[K(PYA)]P[dD]W[HArg]Cᵢᵢᵢ (SEQ ID NO: 7; herein referred to as BCY11815);
Ac-A-[HArg]-D-Cᵢ[HyP][K(PYA)]VNPLCᵢᵢLHP[dD]W[HArg]Cᵢᵢᵢ (SEQ ID NO: 8; herein referred to as BCY11816);
Ac-A-[HArg]-D-Cᵢ[HyP]LVNPLCᵢᵢ[K(PYA)]HP[dD]W[HArg]Cᵢᵢᵢ (SEQ ID NO: 9; herein referred to as BCY11817);
Ac-A-[HArg]-D-Cᵢ[HyP]LVNPLCᵢᵢLKP[dD]W[HArg]Cᵢᵢᵢ (SEQ ID NO: 10; herein referred to as BCY12735);
Ac-A-[HArg]-D-Cᵢ[HyP]KVNPLCᵢᵢLHP[dD]W[HArg]Cᵢᵢ (SEQ ID NO: 11; herein referred to as BCY12736);
Ac-A-[HArg]-D-Cᵢ[HyP]LVNPLCᵢKHP[dD]W[HArg]Cᵢᵢᵢ (SEQ ID NO: 12; herein referred to as BCY12737);
A-[HArg]-D-Cᵢ[HyP]LVNPLCᵢᵢLHP[dE]W[HArg]Cᵢᵢᵢ (SEQ ID NO: 13; herein referred to as BCY12738);
A-[HArg]-E-Cᵢ[HyP]LVNPLCᵢᵢLHP[dE]W[HArg]Cᵢᵢᵢ (SEQ ID NO: 14; herein referred to as BCY12739);
A-[HArg]-D-Cᵢ[HyP]LVNPLCᵢᵢLEP[dD]W[HArg]Cᵢᵢᵢ (SEQ ID NO: 15; herein referred to as BCY12854);
A-[HArg]-D-Cᵢ[HyP]LVNPLCᵢᵢLHP[dD]WTCᵢᵢᵢ (SEQ ID NO: 16; herein referred to as BCY12855);
A-[HArg]-D-Cᵢ[HyP]LVNPLCᵢᵢLEP[dD]WTCᵢᵢᵢ (SEQ ID NO: 17; herein referred to as BCY12856);
A-[HArg]-D-Cᵢ[HyP]LVNPLCᵢᵢLEP[dD]WTCᵢᵢᵢ-[dA] (SEQ ID NO: 18; herein referred to as BCY12857);
Cᵢ[HyP]LVNPLCᵢᵢLEP[dD]WTCᵢᵢᵢ-[dA] (SEQ ID NO: 19; herein referred to as BCY12861);
[NMeAla]-[HArg]-D-Cᵢ[HyP]LVNPLCᵢᵢLEP[dD]WTCᵢᵢᵢ (SEQ ID NO: 20; herein referred to as BCY13122);
[dA]-ED-Cᵢ[HyP]LVNPLCᵢᵢLEP[dD]WTCᵢᵢᵢ (SEQ ID NO: 21; herein referred to as BCY13126);
[dA]-[dA]-D-Cᵢ[HyP]LVNPLCᵢᵢLEP[dD]WTCᵢᵢᵢ (SEQ ID NO: 22; herein referred to as BCY13127);
AD-Cᵢ[HyP]LVNPLCᵢᵢLEP[dD]WTCᵢᵢᵢ (SEQ ID NO: 23; herein referred to as BCY13128);
A-[HArg]-D-Cᵢ[HyP]LVNPLCᵢᵢLEP[dA]WTCᵢᵢᵢ (SEQ ID NO: 24; herein referred to as BCY12858);
Cᵢ[HyP]LVNPLCᵢᵢL[3,3-DPA]P[dD]WTCᵢᵢᵢ (SEQ ID NO: 25; herein referred to as BCY12860);
A-[HArg]-D-Cᵢ[HyP]LVNPLCᵢᵢL[3,3-DPA]P[dD]WTCᵢᵢᵢ (SEQ ID NO: 26; herein referred to as BCY12859);
Ac-Cᵢ[HyP]LVNPLCᵢᵢL[3,3-DPA]P[dD]WTCᵢᵢᵢ-[dK] (SEQ ID NO: 27; herein referred to as BCY13120);
A-[HArg]-D-Cᵢ[HyP][Cba]VNPLCᵢᵢLHP[dD]W[HArg]Cᵢᵢᵢ (SEQ ID NO: 28; herein referred to as BCY12862);
A-[HArg]-D-Cᵢ[HyP][Cba]VNPLCᵢᵢLEP[dD]WTCᵢᵢᵢ (SEQ ID NO: 29; herein referred to as BCY12863);
[dA]-[HArg]-D-Cᵢ[HyP][Cba]VNPLCᵢᵢLEP[dD]WTCᵢᵢᵢ-[dA] (SEQ ID NO: 30; herein referred to as BCY12864);
Cᵢ[HyP][Cba]VNPLCᵢᵢL[3,3-DPA]P[dD]WTCᵢᵢᵢ-[dA] (SEQ ID NO: 31; herein referred to as BCY12865);
A-[HArg]-D-Cᵢ[HyP]LVNPLCᵢᵢL[3,3-DPA]P[dD]W[HArg]Cᵢᵢᵢ (SEQ ID NO: 32; herein referred to as BCY12866);
A-[HArg]-D-Cᵢ[HyP]LVNPLCᵢᵢLHP[d1Nal]W[HArg]Cᵢᵢᵢ (SEQ ID NO: 33; herein referred to as BCY13116);
A-[HArg]-D-Cᵢ[HyP]LVNPLCᵢᵢL[1Nal]P[dD]W[HArg]Cᵢᵢᵢ (SEQ ID NO: 34; herein referred to as BCY13117);
A-[HArg]-D-Cᵢ[HyP]LVNPLCᵢᵢLEP[d1Nal]WTCᵢᵢᵢ (SEQ ID NO: 35; herein referred to as BCY13118);
Cᵢ[HyP]LVNPLCᵢᵢL[1Nal]P[dD]WTCᵢᵢᵢ (SEQ ID NO: 36; herein referred to as BCY13119);
[dA]-[HArg]-D-Cᵢ[HyP][Cba]VNPLCᵢᵢLEP[dA]WTCᵢᵢᵢ-[dA] (SEQ ID NO: 37; herein referred to as BCY13123);
[d1Nal]-[HArg]-D-Cᵢ[HyP][Cba]VNPLCᵢᵢLEP[dA]WTCᵢᵢᵢ-[dA] (SEQ ID NO: 38; herein referred to as BCY13124);
A-[HArg]-D-Cᵢ[HyP][hGlu]VNPLCᵢᵢLHP[dD]W[HArg]Cᵢᵢᵢ (SEQ ID NO: 39; herein referred to as BCY13130);
A-[HArg]-D-Cᵢ[HyP]LVNPLCᵢᵢ[hGlu]HP[dD]W[HArg]Cᵢᵢᵢ (SEQ ID NO: 40; herein referred to as BCY13131);
A-[HArg]-D-Cᵢ[HyP]LVNPLCᵢᵢL[hGlu]P[dD]W[HArg]Cᵢᵢᵢ (SEQ ID NO: 41; herein referred to as BCY13132);
A-[HArg]-D-Cᵢ[HyP]LVNPLCᵢᵢLHP[dNle]W[HArg]Cᵢᵢᵢ (SEQ ID NO: 42; herein referred to as BCY13134);
A-[HArg]-D-Cᵢ[HyP]LVNPLCᵢᵢL[Nle]P[dD]W[HArg]Cᵢᵢᵢ (SEQ ID NO: 43; herein referred to as BCY13135);
A[HArg]DCᵢ[HyP]LVNPLCᵢᵢLHP[dD]W[HArg][Cysam]ᵢᵢᵢ (SEQ ID NO: 44; herein referred to as BCY13133);
[Ac] Cᵢ[HyP]LVNPLCᵢᵢLHP[dD]W[HArg]CᵢᵢᵢL[dH]G[dK] (SEQ ID NO: 45; herein referred to as BCY13125);
[MerPro]ᵢ[HyP]LVNPLCᵢᵢL[3,3-DPA]P[dD]WTCᵢᵢᵢ[dK] (SEQ ID NO: 46; herein referred to as BCY13129);
A[HArg]DCᵢ[HyP]LVNPLCᵢᵢL[His3Me]P[dD]W[HArg]Cᵢᵢ (SEQ ID NO: 47; herein referred to as BCY13917);
A[HArg]DCᵢ[HyP]LVNPLCᵢᵢL[His1Me]P[dD]W[HArg]Cᵢᵢ (SEQ ID NO: 48; herein referred to as BCY13918);
A[HArg]DCᵢ[HyP]LVNPLCᵢᵢL[4ThiAz]P[dD]W[HArg]Cᵢᵢᵢ (SEQ ID NO: 49; herein referred to as BCY13919);
A[HArg]DCᵢ[HyP]LVNPLCᵢᵢLFP[dD]W[HArg]Cᵢᵢ (SEQ ID NO: 50; herein referred to as BCY13920);
A[HArg]DCᵢ[HyP]LVNPLCᵢᵢL[Thi]P[dD]W[HArg]Cᵢᵢ (SEQ ID NO: 51; herein referred to as BCY13922);
A[HArg]DCᵢ[HyP]LVNPLCᵢᵢL[3Thi]P[dD]W[HArg]Cᵢᵢ (SEQ ID NO: 52; herein referred to as BCY13923);
A[HArg]DCᵢ[HyP]LVNPLCᵢLNP[dD]W[HArg]Cᵢᵢᵢ (SEQ ID NO: 53; herein referred to as BCY14047);
A[HArg]DCᵢ[HyP]LVNPLCᵢLQP[dD]W[HArg]Cᵢᵢ (SEQ ID NO: 54; herein referred to as BCY14048);
A[HArg]DCᵢ[HyP]LVNPLCᵢᵢL[K(PYA)]P[dD]W[HArg]Cᵢᵢ (SEQ ID NO: 55; herein referred to as BCY14311);
[PYA]A[HArg]DCᵢ[HyP]LVNPLCᵢᵢLKP[dD]W[HArg]Cᵢᵢ (SEQ ID NO: 56; herein referred to as BCY14312);
A[HArg]DCᵢ[HyP]LVNPLCᵢᵢL[K(PYA-(Palmitoyl-Glu-LysN₃)]P[dD]W[HArg]Cᵢᵢ (SEQ ID NO: 57; herein referred to as BCY14313);
(Palmitoyl-Glu-LysN₃)[PYA]A[HArg]DCᵢ[HyP]LVNPLCᵢLKP[dD]W[HArg]Cᵢᵢ (SEQ ID NO: 58; herein referred to as BCY14327); and
A[HArg]DCᵢ[HyP]LVNPLCᵢᵢL[pCoPhe]P[dD]W[HArg]Cᵢᵢ (SEQ ID NO: 59; herein referred to as BCY14462);
   wherein Ac represents acetyl, HyP represents hydroxyproline, HArg represents homoarginine, PYA represents 4-pentynoic acid, 3,3-DPA represents 3,3-diphenylalanine, Cba represents β-cyclobutylalanine, 1Nal represents 1-naphthylalanine, NMeAla represents N-methyl-alanine, His1Me represents N1-methyl-L-histidine, His3Me represents N3-methyl-L-histidine, 4ThiAz represents beta-(4-thiazolyl)-alanine, Thi represents 2-thienyl-alanine, 3Thi represents 3-thienylalanine, Palmitoyl-Glu-LysN₃ represents N2-((S)-4-carboxy-4-palmitamidobutanoyl)-N6-diazo-L-lysine:

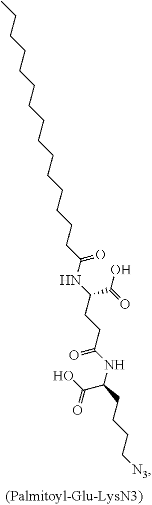 [SMILES: CCCCCCCCCCCCCCCC(=O)N[C@@H](CCC(=O)N[C@@H](CCCCN=[N+]=[N-])C(=O)O)C(=O)O]

pCoPhe represents para-carboxy-phenylalanine, hGlu represents homoglutamic acid, B-Ala represents beta-alanine, Sar₁₀ represents 10 sarcosine units, Nle represents norleucine, and [MerPro]ᵢ, Cᵢ, Cᵢᵢ, Cᵢᵢᵢ and [Cysam]ᵢᵢᵢ represent first (i), second (ii) and third (iii) reactive groups which are selected from cysteine, 3-mercaptopropionic acid (MerPro) and cysteamine (Cysam), or a pharmaceutically acceptable salt thereof.

According to a further aspect of the invention, there is provided a pharmaceutical composition comprising a peptide ligand as defined herein in combination with one or more pharmaceutically acceptable excipients.

According to a further aspect of the invention, there is provided a peptide ligand as defined herein for use in preventing, suppressing or treating cancer.

### DETAILED DESCRIPTION OF THE INVENTION

According to a first aspect of the invention, there is provided a peptide ligand specific for EphA2 comprising a polypeptide comprising at least three reactive groups, separated by at least two loop sequences, and a molecular scaffold, which is 1,1′,1″-(1,3,5-triazinane-1,3,5-triyl)triprop-2-en-1-one, which forms covalent bonds with the reactive groups of the polypeptide such that at least two polypeptide loops are formed on the molecular scaffold, wherein the peptide ligand comprises an amino acid sequence selected from:
A-[HArg]-D-Cᵢ[HyP]LVNPLCᵢᵢLHP[dD]W[HArg]Cᵢᵢ (SEQ ID NO: 1; herein referred to as BCY9594);
[PYA]-A-[HArg]-D-Cᵢ[HyP]LVNPLCᵢᵢLHP[dD]W[HArg]Cᵢᵢᵢ (SEQ ID NO: 2; herein referred to as BCY11813);
Ac-A-[HArg]-D-Cᵢ[HyP]LVNPLCᵢᵢLHP[dD]W[HArg]Cᵢᵢᵢ)-[K(PYA)] (SEQ ID NO: 3; herein referred to as BCY11814);
Ac-A-[HArg]-D-Cᵢ[HyP]LVNPLCᵢᵢLHP[dD]W[HArg]Cᵢᵢᵢ-K (SEQ ID NO: 4; herein referred to as BCY12734);
[NMeAla]-[HArg]-D-Cᵢ[HyP]LVNPLCᵢᵢLHP[dD]W[HArg]Cᵢᵢᵢ (SEQ ID NO: 5; herein referred to as BCY13121);
[PYA]-[B-Ala]-[Sar₁₀]-VGP-CᵢLWDPTPCᵢᵢANLHL[HArg]Cᵢᵢᵢ (SEQ ID NO: 6; herein referred to as BCY8941);
Ac-A-[HArg]-D-Cᵢ[HyP]LVNPLCᵢᵢL[K(PYA)]P[dD]W[HArg]Cᵢᵢᵢ (SEQ ID NO: 7; herein referred to as BCY11815);
Ac-A-[HArg]-D-Cᵢ[HyP][K(PYA)]VNPLCᵢᵢLHP[dD]W[HArg]Cᵢᵢᵢ (SEQ ID NO: 8; herein referred to as BCY11816);
Ac-A-[HArg]-D-Cᵢ[HyP]LVNPLCᵢᵢ[K(PYA)]HP[dD]W[HArg]Cᵢᵢᵢ (SEQ ID NO: 9; herein referred to as BCY11817);
Ac-A-[HArg]-D-Cᵢ[HyP]LVNPLCᵢLKP[dD]W[HArg]Cᵢᵢᵢ (SEQ ID NO: 10; herein referred to as BCY12735);
Ac-A-[HArg]-D-Cᵢ[HyP]KVNPLCᵢᵢLHP[dD]W[HArg]Cᵢᵢᵢ (SEQ ID NO: 11; herein referred to as BCY12736);
Ac-A-[HArg]-D-Cᵢ[HyP]LVNPLCᵢᵢKHP[dD]W[HArg]Cᵢᵢᵢ (SEQ ID NO: 12; herein referred to as BCY12737);
A-[HArg]-D-Cᵢ[HyP]LVNPLCᵢᵢLHP[dE]W[HArg]Cᵢᵢᵢ (SEQ ID NO: 13; herein referred to as BCY12738);
A-[HArg]-E-Cᵢ[HyP]LVNPLCᵢᵢLHP[dE]W[HArg]Cᵢᵢᵢ (SEQ ID NO: 14; herein referred to as BCY12739);
A-[HArg]-D-Cᵢ[HyP]LVNPLCᵢᵢLEP[dD]W[HArg]Cᵢᵢᵢ (SEQ ID NO: 15; herein referred to as BCY12854);
A-[HArg]-D-Cᵢ[HyP]LVNPLCᵢᵢLHP[dD]WTCᵢᵢᵢ (SEQ ID NO: 16; herein referred to as BCY12855);
A-[HArg]-D-Cᵢ[HyP]LVNPLCᵢᵢLEP[dD]WTCᵢᵢᵢ (SEQ ID NO: 17; herein referred to as BCY12856);
A-[HArg]-D-Cᵢ[HyP]LVNPLCᵢᵢLEP[dD]WTCᵢᵢᵢ-[dA] (SEQ ID NO: 18; herein referred to as BCY12857);
Cᵢ[HyP]LVNPLCᵢᵢLEP[dD]WTCᵢᵢ-[dA] (SEQ ID NO: 19; herein referred to as BCY12861);
[NMeAla]-[HArg]-D-Cᵢ[HyP]LVNPLCᵢᵢLEP[dD]WTCᵢᵢᵢ (SEQ ID NO: 20; herein referred to as BCY13122);
[dA]-ED-Cᵢ[HyP]LVNPLCᵢᵢLEP[dD]WTCᵢᵢᵢ (SEQ ID NO: 21; herein referred to as BCY13126);
[dA]-[dA]-D-Cᵢ[HyP]LVNPLCᵢᵢLEP[dD]WTCᵢᵢᵢ (SEQ ID NO: 22; herein referred to as BCY13127);
AD-Cᵢ[HyP]LVNPLCᵢᵢLEP[dD]WTCᵢᵢᵢ (SEQ ID NO: 23; herein referred to as BCY13128);
A-[HArg]-D-Cᵢ[HyP]LVNPLCᵢᵢLEP[dA]WTCᵢᵢᵢ (SEQ ID NO: 24; herein referred to as BCY12858);
Cᵢ[HyP]LVNPLCᵢᵢL[3,3-DPA]P[dD]WTCᵢᵢᵢ (SEQ ID NO: 25; herein referred to as BCY12860);
A-[HArg]-D-Cᵢ[HyP]LVNPLCᵢᵢL[3,3-DPA]P[dD]WTCᵢᵢᵢ (SEQ ID NO: 26; herein referred to as BCY12859);
Ac-Cᵢ[HyP]LVNPLCᵢᵢL[3,3-DPA]P[dD]WTCᵢᵢᵢ-[dK] (SEQ ID NO: 27; herein referred to as BCY13120);
A-[HArg]-D-Cᵢ[HyP][Cba]VNPLCᵢᵢLHP[dD]W[HArg]Cᵢᵢᵢ (SEQ ID NO: 28; herein referred to as BCY12862);
A-[HArg]-D-Cᵢ[HyP][Cba]VNPLCᵢᵢLEP[dD]WTCᵢᵢᵢ (SEQ ID NO: 29; herein referred to as BCY12863);
[dA]-[HArg]-D-Cᵢ[HyP][Cba]VNPLCᵢᵢLEP[dD]WTCᵢᵢᵢ-[dA] (SEQ ID NO: 30; herein referred to as BCY12864);
Cᵢ[HyP][Cba]VNPLCᵢᵢL[3,3-DPA]P[dD]WTCᵢᵢᵢ-[dA] (SEQ ID NO: 31; herein referred to as BCY12865);
A-[HArg]-D-Cᵢ[HyP]LVNPLCᵢᵢL[3,3-DPA]P[dD]W[HArg]Cᵢᵢᵢ (SEQ ID NO: 32; herein referred to as BCY12866);
A-[HArg]-D-Cᵢ[HyP]LVNPLCᵢᵢLHP[d1Nal]W[HArg]Cᵢᵢᵢ (SEQ ID NO: 33; herein referred to as BCY13116);
A-[HArg]-D-Cᵢ[HyP]LVNPLCᵢᵢL[1Nal]P[dD]W[HArg]Cᵢᵢᵢ (SEQ ID NO: 34; herein referred to as BCY13117);
A-[HArg]-D-Cᵢ[HyP]LVNPLCᵢᵢLEP[d1Nal]WTCᵢᵢᵢ (SEQ ID NO: 35; herein referred to as BCY13118);
Cᵢ[HyP]LVNPLCᵢᵢL[1Nal]P[dD]WTCᵢᵢᵢ (SEQ ID NO: 36; herein referred to as BCY13119);
[dA]-[HArg]-D-Cᵢ[HyP][Cba]VNPLCᵢᵢLEP[dA]WTCᵢᵢᵢ-[dA] (SEQ ID NO: 37; herein referred to as BCY13123);
[d1Nal]-[HArg]-D-Cᵢ[HyP][Cba]VNPLCᵢᵢLEP[dA]WTCᵢᵢᵢ-[dA] (SEQ ID NO: 38; herein referred to as BCY13124);
A-[HArg]-D-Cᵢ[HyP][hGlu]VNPLCᵢᵢLHP[dD]W[HArg]Cᵢᵢᵢ (SEQ ID NO: 39; herein referred to as BCY13130);
A-[HArg]-D-Cᵢ[HyP]LVNPLCᵢᵢ[hGlu]HP[dD]W[HArg]Cᵢᵢᵢ (SEQ ID NO: 40; herein referred to as BCY13131);
A-[HArg]-D-Cᵢ[HyP]LVNPLCᵢᵢL[hGlu]P[dD]W[HArg]Cᵢᵢᵢ (SEQ ID NO: 41; herein referred to as BCY13132);
A-[HArg]-D-Cᵢ[HyP]LVNPLCᵢᵢLHP[dNle]W[HArg]Cᵢᵢᵢ (SEQ ID NO: 42; herein referred to as BCY13134);
A-[HArg]-D-Cᵢ[HyP]LVNPLCᵢᵢL[Nle]P[dD]W[HArg]Cᵢᵢᵢ (SEQ ID NO: 43; herein referred to as BCY13135);
A[HArg]DCᵢ[HyP]LVNPLCᵢᵢLHP[dD]W[HArg][Cysam]ᵢᵢᵢ (SEQ ID NO: 44; herein referred to as BCY13133);
[Ac] Cᵢ[HyP]LVNPLCᵢᵢLHP[dD]W[HArg]CᵢᵢᵢL[dH]G[dK] (SEQ ID NO: 45; herein referred to as BCY13125);
[MerPro]: [HyP]LVNPLCᵢᵢL[3,3-DPA]P[dD]WTCᵢᵢᵢ[dK] (SEQ ID NO: 46; herein referred to as BCY13129);
A[HArg]DCᵢ[HyP]LVNPLCᵢᵢL[His3Me]P[dD]W[HArg]Cᵢᵢᵢ (SEQ ID NO: 47; herein referred to as BCY13917);
A[HArg]DCᵢ[HyP]LVNPLCᵢᵢL[His1Me]P[dD]W[HArg]Cᵢᵢᵢ (SEQ ID NO: 48; herein referred to as BCY13918);
A[HArg]DCᵢ[HyP]LVNPLCᵢᵢL[4ThiAz]P[dD]W[HArg]Cᵢᵢᵢ (SEQ ID NO: 49; herein referred to as BCY13919);
A[HArg]DCᵢ[HyP]LVNPLCᵢᵢLFP[dD]W[HArg]Cᵢᵢᵢ (SEQ ID NO: 50; herein referred to as BCY13920);
A[HArg]DCᵢ[HyP]LVNPLCᵢᵢL[Thi]P[dD]W[HArg]Cᵢᵢᵢ (SEQ ID NO: 51; herein referred to as BCY13922);
A[HArg]DCᵢ[HyP]LVNPLCᵢᵢL[3Thi]P[dD]W[HArg]Cᵢᵢᵢ (SEQ ID NO: 52; herein referred to as BCY13923);
A[HArg]DCᵢ[HyP]LVNPLCᵢLNP[dD]W[HArg]Cᵢᵢᵢ (SEQ ID NO: 53; herein referred to as BCY14047);
A[HArg]DCᵢ[HyP]LVNPLCᵢLQP[dD]W[HArg]Cᵢᵢᵢ (SEQ ID NO: 54; herein referred to as BCY14048);
A[HArg]DCᵢ[HyP]LVNPLCᵢᵢL[K(PYA)]P[dD]W[HArg]Cᵢᵢᵢ (SEQ ID NO: 55; herein referred to as BCY14311);
[PYA]A[HArg]DCᵢ[HyP]LVNPLCᵢᵢLKP[dD]W[HArg]Cᵢᵢᵢ (SEQ ID NO: 56; herein referred to as BCY14312);
A[HArg]DCᵢ[HyP]LVNPLCᵢᵢL[K(PYA-(Palmitoyl-Glu-LysN₃)]P[dD]W[HArg]Cᵢᵢᵢ (SEQ ID NO: 57; herein referred to as BCY14313);
(Palmitoyl-Glu-LysN₃)[PYA]A[HArg]DCᵢ[HyP]LVNPLCᵢLKP[dD]W[HArg]Cᵢᵢᵢ (SEQ ID NO: 58; herein referred to as BCY14327); and
A[HArg]DCᵢ[HyP]LVNPLCᵢᵢL[pCoPhe]P[dD]W[HArg]Cᵢᵢᵢ (SEQ ID NO: 59; herein referred to as BCY14462);
   wherein Ac represents acetyl, HyP represents hydroxyproline, HArg represents homoarginine, PYA represents 4-pentynoic acid, 3,3-DPA represents 3,3-diphenylalanine, Cba represents β-cyclobutylalanine, 1Nal represents 1-naphthylalanine, NMeAla represents N-methyl-alanine, His1Me represents N1-methyl-L-histidine, His3Me represents N3-methyl-L-histidine, 4ThiAz represents beta-(4-thiazolyl)-alanine, Thi represents 2-thienyl-alanine, 3Thi represents 3-thienylalanine, Palmitoyl-Glu-LysN₃ represents N2-((S)-4-carboxy-4-palmitamidobutanoyl)-N6-diazo-L-lysine:

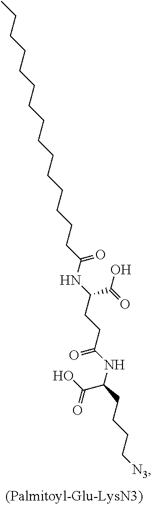 [SMILES: CCCCCCCCCCCCCCCC(=O)N[C@@H](CCC(=O)N[C@@H](CCCCN=[N+]=[N-])C(=O)O)C(=O)O]

pCoPhe represents para-carboxy-phenylalanine, hGlu represents homoglutamic acid, B-Ala represents beta-alanine, Sar₁₀ represents 10 sarcosine units, Nle represents norleucine, and [MerPro]ᵢ, Cᵢ, Cᵢᵢ, Cᵢᵢᵢ and [Cysam]ᵢᵢᵢ represent first (i), second (ii) and third (iii) reactive groups which are selected from cysteine, 3-mercaptopropionic acid (MerPro) and cysteamine (Cysam), or a pharmaceutically acceptable salt thereof.

In one particular embodiment, the EphA2 binding bicyclic peptide ligand is selected from: BCY13118, BCY12860, BCY12859, BCY13119, BCY13917, BCY13918, BCY13919, BCY13920, BCY13922, BCY13923, BCY14047, BCY14048, BCY13135, BCY12865, BCY13120 and BCY13117.

In one particular embodiment, the EphA2 binding bicyclic peptide ligand is BCY13118 or a pharmaceutically acceptable salt thereof.

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, NY; Ausubel et al., Short Protocols in Molecular Biology (1999) 4^{th} ed., John Wiley & Sons, Inc.), which are incorporated herein by reference.

### Nomenclature

### Numbering

When referring to amino acid residue positions within compounds of the invention, cysteine residues (Cᵢ, Cᵢᵢ, and Cᵢᵢᵢ) are omitted from the numbering as they are invariant, therefore, the numbering of amino acid residues within SEQ ID NO: 1 is referred to as below:

(SEQ ID NO: 1)
A-[HArg]-D-Cᵢ-[HyP]₁-L₂-V₃-N₄-P₅-L₆-Cᵢᵢ-
L₇-H₈-P₉-[dD]₁₀-W11-[HArg]₁₂-Cᵢᵢᵢ.

For the purpose of this description, all bicyclic peptides are assumed to be cyclised with 1,1′,1″-(1,3,5-triazinane-1,3,5-triyl)triprop-2-en-1-one (TATA) and yielding a tri-substituted structure. Cyclisation with TATA occurs on Cᵢ, Cᵢᵢ, and Cᵢᵢᵢ.

### Molecular Format

N- or C-terminal extensions to the bicycle core sequence are added to the left or right side of the sequence, separated by a hyphen. For example, an N-terminal βAla-Sar10-Ala tail would be denoted as:
βAla-Sar10-A

### Inversed Peptide Sequences

In light of the disclosure in Nair et al (2003) J Immunol 170 (3), 1362-1373, it is envisaged that the peptide sequences disclosed herein would also find utility in their retro-inverso form.

For example, the sequence is reversed (i.e. N-terminus becomes C-terminus and vice versa) and their stereochemistry is likewise also reversed (i.e. D-amino acids become L-amino acids and vice versa). For the avoidance of doubt, references to amino acids either as their full name or as their amino acid single or three letter codes are intended to be represented herein as L-amino acids unless otherwise stated. If such an amino acid is intended to be represented as a D-amino acid then the amino acid will be prefaced with a lower case d within square parentheses, for example [dA], [dD], [dE], [dK], [d1Nal], [dNle], etc.

### Advantages of the Peptide Ligands

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. This is a typical requirement for preclinical pharmacodynamics and pharmacokinetic evaluation;
Protease stability. Bicyclic peptide ligands should in most circumstances demonstrate stability to plasma proteases, epithelial (“membrane-anchored”) proteases, gastric and intestinal proteases, lung surface proteases, intracellular proteases and the like. Protease stability should be maintained between different species such that a bicyclic peptide lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes;
An optimal plasma half-life in the circulation. Depending upon the clinical indication and treatment regimen, it may be required to develop a bicyclic peptide with short or prolonged in vivo exposure times for the management of either chronic or acute disease states. The optimal exposure time will be governed by the requirement for sustained exposure (for maximal therapeutic efficiency) versus the requirement for short exposure times to minimise toxicological effects arising from sustained exposure to the agent;
Selectivity. Certain peptide ligands of the invention demonstrate good selectivity over other Eph receptor tyrosine kinases, such as EphA1, EphA3, EphA4, EphA5, EphA6, EphA7 and EphB1 and factor XIIA, carbonic anhydrase 9 and CD38. It should also be noted that selected peptide ligands of the invention exhibit cross reactivity with other species (eg mouse and rat) to permit testing in animal models; and
Safety. Bleeding events have been reported in pre-clinical in vivo models and clinical trials with EphA2 Antibody Drug Conjugates. For example, a phase 1, open-label study with MEDI-547 was halted due to bleeding and coagulation events that occurred in 5 of 6 patients (Annunziata et al, Invest New Drugs (2013) 31:77-84). The bleeding events observed in patients were consistent with effects on the coagulation system observed in rat and monkey pre-clinical studies: increased activated partial thromboplastin time and increased fibrinogen/fibrin degradation product (Annunziata et al IBID). Overt bleeding events were reportedly seen in toxicology studies in monkeys (Annunziata et al, IBID). Taken together these results imply that MEDI-547 causes Disseminated Intravascular Coagulation (DIC) in both preclinical species and patients.

### Peptide Ligands

A peptide ligand, as referred to herein, refers to a peptide covalently bound to a molecular scaffold. Typically, such peptides comprise two or more reactive groups (i.e. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three reactive groups selected from cysteine, 3-mercaptopropionic acid and/or cysteamine and form at least two loops on the scaffold.

### Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH⁴⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the invention contain an amine function, these may form quaternary ammonium salts, for example by reaction with an alkylating agent according to methods well known to the skilled person. Such quaternary ammonium compounds are within the scope of the invention.

### Reactive Groups

The molecular scaffold of the invention may be bonded to the polypeptide via functional or reactive groups on the polypeptide. These are typically formed from the side chains of particular amino acids found in the polypeptide polymer. Such reactive groups may be a cysteine side chain, a lysine side chain, or an N-terminal amine group or any other suitable reactive group, such as penicillamine. Details of suitable reactive groups may be found in WO 2009/098450.

Examples of reactive groups of natural amino acids are the thiol group of cysteine, the amino group of lysine, the carboxyl group of aspartate or glutamate, the guanidinium group of arginine, the phenolic group of tyrosine or the hydroxyl group of serine. Non-natural amino acids can provide a wide range of reactive groups including an azide, a keto-carbonyl, an alkyne, a vinyl, or an aryl halide group. The amino and carboxyl group of the termini of the polypeptide can also serve as reactive groups to form covalent bonds to a molecular scaffold/molecular core.

The polypeptides of the invention contain at least three reactive groups. Said polypeptides can also contain four or more reactive groups. The more reactive groups are used, the more loops can be formed in the molecular scaffold.

In a preferred embodiment, polypeptides with three reactive groups are generated. Reaction of said polypeptides with a molecular scaffold/molecular core having a three-fold rotational symmetry generates a single product isomer. The generation of a single product isomer is favourable for several reasons. The nucleic acids of the compound libraries encode only the primary sequences of the polypeptide but not the isomeric state of the molecules that are formed upon reaction of the polypeptide with the molecular core. If only one product isomer can be formed, the assignment of the nucleic acid to the product isomer is clearly defined. If multiple product isomers are formed, the nucleic acid cannot give information about the nature of the product isomer that was isolated in a screening or selection process. The formation of a single product isomer is also advantageous if a specific member of a library of the invention is synthesized. In this case, the chemical reaction of the polypeptide with the molecular scaffold yields a single product isomer rather than a mixture of isomers.

In another embodiment, polypeptides with four reactive groups are generated. Reaction of said polypeptides with a molecular scaffold/molecular core having a tetrahedral symmetry generates two product isomers. Even though the two different product isomers are encoded by one and the same nucleic acid, the isomeric nature of the isolated isomer can be determined by chemically synthesizing both isomers, separating the two isomers and testing both isomers for binding to a target ligand.

In one embodiment of the invention, at least one of the reactive groups of the polypeptides is orthogonal to the remaining reactive groups. The use of orthogonal reactive groups allows the directing of said orthogonal reactive groups to specific sites of the molecular core. Linking strategies involving orthogonal reactive groups may be used to limit the number of product isomers formed. In other words, by choosing distinct or different reactive groups for one or more of the at least three bonds to those chosen for the remainder of the at least three bonds, a particular order of bonding or directing of specific reactive groups of the polypeptide to specific positions on the molecular scaffold may be usefully achieved.

In another embodiment, the reactive groups of the polypeptide of the invention are reacted with molecular linkers wherein said linkers are capable to react with a molecular scaffold so that the linker will intervene between the molecular scaffold and the polypeptide in the final bonded state.

In some embodiments, amino acids of the members of the libraries or sets of polypeptides can be replaced by any natural or non-natural amino acid. Excluded from these exchangeable amino acids are the ones harbouring functional groups for cross-linking the polypeptides to a molecular core, such that the loop sequences alone are exchangeable. The exchangeable polypeptide sequences have either random sequences, constant sequences or sequences with random and constant amino acids. The amino acids with reactive groups are either located in defined positions within the polypeptide, since the position of these amino acids determines loop size.

In one embodiment, a polypeptide with three reactive groups has the sequence (X)ₗY(X)ₘY(X)ₙY(X)ₒ, wherein Y represents an amino acid with a reactive group, X represents a random amino acid, m and n are numbers between 3 and 6 defining the length of intervening polypeptide segments, which may be the same or different, and l and o are numbers between 0 and 20 defining the length of flanking polypeptide segments.

Alternatives to thiol-mediated conjugations can be used to attach the molecular scaffold to the peptide via covalent interactions. Alternatively these techniques may be used in modification or attachment of further moieties (such as small molecules of interest which are distinct from the molecular scaffold) to the polypeptide after they have been selected or isolated according to the present invention—in this embodiment then clearly the attachment need not be covalent and may embrace non-covalent attachment. These methods may be used instead of (or in combination with) the thiol mediated methods by producing phage that display proteins and peptides bearing unnatural amino acids with the requisite chemical reactive groups, in combination small molecules that bear the complementary reactive group, or by incorporating the unnatural amino acids into a chemically or recombinantly synthesised polypeptide when the molecule is being made after the selection/isolation phase. Further details can be found in WO 2009/098450 or Heinis et al., Nat Chem Biol 2009, 5 (7), 502-7.

In one embodiment, the reactive groups are selected from cysteine, 3-mercaptopropionic acid and/or cysteamine residues.

### Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal cysteine group (the group referred to herein as Cᵢ) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal cysteine group (the group referred to herein as Cᵢᵢᵢ) is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, Cα-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine (Cᵢ) and/or the C-terminal cysteine (Cᵢᵢ).

In one embodiment, the modified derivative comprises replacement of one or more oxidation sensitive amino acid residues with one or more oxidation resistant amino acid residues. In a further embodiment, the modified derivative comprises replacement of a tryptophan residue with a naphthylalanine or alanine residue. This embodiment provides the advantage of improving the pharmaceutical stability profile of the resultant bicyclic peptide ligand.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise β-turn conformations (Tugyi et al (2005) PNAS, 102 (2), 413-418).

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.
   (for reviews see Gentilucci et al, Curr. Pharmaceutical Design, (2010), 16, 3185-203, and Nestor et al, Curr. Medicinal Chem (2009), 16, 4399-418).

### Isotopic Variations

The present invention includes all pharmaceutically acceptable (radio) isotope-labeled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio) isotopes, and peptide ligands of the invention, wherein certain functional groups are covalently replaced with relevant (radio) isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies, and to clinically assess the presence and/or absence of the Nectin-4 target on diseased tissues. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labeled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

### Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al (supra).

Thus, the invention also relates to manufacture of polypeptides or conjugates selected as set out herein, wherein the manufacture comprises optional further steps as explained below. In one embodiment, these steps are carried out on the end product polypeptide/conjugate made by chemical synthesis.

Optionally amino acid residues in the polypeptide of interest may be substituted when manufacturing a conjugate or complex.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio) conjugation techniques may be used to introduce an activated or activatable N- or C-terminus. Alternatively additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al. Proc Natl Acad Sci USA. 1994 Dec. 20; 91 (26): 12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptides to dissociate from each other once within the reducing environment of the cell. In this case, the molecular scaffold (e.g. TATA) could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptides, forming a disulfide-linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

### Pharmaceutical Compositions

According to a further aspect of the invention, there is provided a pharmaceutical composition comprising a peptide ligand as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The peptide ligands of the present invention may be used as separately administered compositions or in conjunction with other agents. These can include antibodies, antibody fragments and various immunotherapeutic drugs, such as cylcosporine, methotrexate, adriamycin or cisplatinum and immunotoxins. Pharmaceutical compositions can include “cocktails” of various cytotoxic or other agents in conjunction with the protein ligands of the present invention, or even combinations of selected polypeptides according to the present invention having different specificities, such as polypeptides selected using different target ligands, whether or not they are pooled prior to administration.

The route of administration of pharmaceutical compositions according to the invention may be any of those commonly known to those of ordinary skill in the art. For therapy, the peptide ligands of the invention can be administered to any patient in accordance with standard techniques. The administration can be by any appropriate mode, including parenterally, intravenously, intramuscularly, intraperitoneally, transdermally, via the pulmonary route, or also, appropriately, by direct infusion with a catheter. Preferably, the pharmaceutical compositions according to the invention will be administered by inhalation. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for prophylactic and/or therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 0.005 to 5.0 mg of selected peptide ligand per kilogram of body weight, with doses of 0.05 to 2.0 mg/kg/dose being more commonly used. For prophylactic applications, compositions containing the present peptide ligands or cocktails thereof may also be administered in similar or slightly lower dosages.

A composition containing a peptide ligand according to the present invention may be utilised in prophylactic and therapeutic settings to aid in the alteration, inactivation, killing or removal of a select target cell population in a mammal. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

### Therapeutic Uses

According to a further aspect of the invention, there is provided a heterotandem bicyclic peptide complex as defined herein for use in preventing, suppressing or treating cancer.

Examples of cancers (and their benign counterparts) which may be treated (or inhibited) include, but are not limited to tumors of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenousleukemia [AML], chronic myelogenousleukemia [CML], chronic myelomonocyticleukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocyticleukemia); tumors of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumors, benign and malignant histiocytomas, and dermatofibrosarcomaprotuberans; tumors of the central or peripheral nervous system (for example astrocytomas, gliomas and glioblastomas, meningiomas, ependymomas, pineal tumors and schwannomas); endocrine tumors (for example pituitary tumors, adrenal tumors, islet cell tumors, parathyroid tumors, carcinoid tumors and medullary carcinoma of the thyroid); ocular and adnexal tumors (for example retinoblastoma); germ cell and trophoblastic tumors (for example teratomas, seminomas, dysgerminomas, hydatidiform moles and choriocarcinomas); and paediatric and embryonal tumors (for example medulloblastoma, neuroblastoma, Wilms tumor, and primitive neuroectodermal tumors); or syndromes, congenital or otherwise, which leave the patient susceptible to malignancy (for example Xeroderma Pigmentosum).

In a further embodiment, the cancer is selected from a hematopoietic malignancy such as selected from: non-Hodgkin's lymphoma (NHL), Burkitt's lymphoma (BL), multiple myeloma (MM), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), and chronic myeloid leukemia (CML).

References herein to the term “prevention” involves administration of the protective composition prior to the induction of the disease. “Suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available. The use of animal model systems is facilitated by the present invention, which allows the development of polypeptide ligands which can cross react with human and animal targets, to allow the use of animal models.

The invention is further described below with reference to the following examples.

### EXAMPLES

### Materials and Methods

### Peptide Synthesis

Peptides were synthesized by solid phase synthesis. Rink Amide MBHA Resin was used. To a mixture containing Rink Amide MBHA (0.4-0.45 mmol/g) and Fmoc-Cys(Trt)-OH (3.0 eq) was added DMF, then DIC (3 eq) and HOAt (3 eq) were added and mixed for 1 hour. 20% piperidine in DMF was used for deblocking. Each subsequent amino acid was coupled with 3 eq using activator reagents, DIC (3.0 eq) and HOAT (3.0 eq) in DMF. The reaction was monitored by ninhydrin color reaction or tetrachlor color reaction. After synthesis completion, the peptide resin was washed with DMF×3, MeOH×3, and then dried under N₂ bubbling overnight. The peptide resin was then treated with 92.5% TFA/2.5% TIS/2.5% EDT/2.5% H₂O for 3 h. The peptide was precipitated with cold isopropyl ether and centrifuged (3 min at 3000 rpm). The pellet was washed twice with isopropyl ether and the crude peptide was dried under vacuum for 2 hours and then lyophilised. The lyophilised powder was dissolved in of ACN/H₂O (50:50), and a solution of 100 mM TATA in ACN was added, followed by ammonium bicarbonate in H₂O (1M) and the solution mixed for 1 h. Once the cyclisation was complete, the reaction was quenched with 1M aq. Cysteine hydrochloride (10 eq relative to TATA), then mixed and left to stand for an hour. The solution was lyophilised to afford crude product. The crude peptide was purified by Preparative HPLC and lyophilized to give the product

All amino acids, unless noted otherwise, were used in the L-configurations.

### Biological Data

Peptides without a fluorescent tag were tested in competition with a peptide with a fluorescent tag and a known Kd. The fluorescent tracer used was BCY90 (Kd=2 nM), sequence [FI]G[Sar]₅ADVTCPWGPFWCPVNRPGCA-CONH₂ (SEQ ID NO: 60) where FI is 5/6-carboxfluorescein and Sar is sarcosine.

Peptides were diluted to an appropriate concentration in assay buffer as described in the direct binding assay with a maximum of 5% DMSO, then serially diluted 1 in 2. Five μL of diluted peptide was added to the plate followed by 10 μL of human EphA2 at a concentration of 25 nM, then 10 μL fluorescent peptide added (final concentration 0.8 nM). Measurements were conducted, however the gain was determined prior to the first measurement. Data analysis was in Systat Sigmaplot version 12.0 where the mP values were fit to a user defined cubic equation to generate a Ki value:
f=ymax+(ymin−ymax)/Lig*((Lig*((2*((Klig+Kcomp+Lig+Comp−Prot*c)\hat{ }2−3*(Kcomp*(Lig−Prot*c)+Klig*(Comp−Prot*c)+Klig*Kcomp))\hat{ }0.5*COS(ARCCOS((−2*(Klig+Kcomp+Lig+Comp−Prot*c)\hat{ }3+9*(Klig+Kcomp+Lig+Comp−Prot*c)*(Kcomp*(Lig−Prot*c)+Klig*(Comp−Prot*c)+Klig*Kcomp)−27*(−1*Klig*Kcomp*Prot*c))/(2*((((Klig+Kcomp+Lig+Comp−Prot*c)\hat{ }2−3*(Kcomp*(Lig−Prot*c)+Klig*(Comp−Prot*c)+Klig*Kcomp))\hat{ }3)\hat{ }0.5)))/3))−(Klig+Kcomp+Lig+Comp−Prot*c)))/((3*Klig)+((2*((Klig+Kcomp+Lig+Comp−Prot*c)\hat{ }2−3*(Kcomp*(Lig−Prot*c)+Klig*(Comp−Prot*c)+Klig*Kcomp))\hat{ }0.5*COS(ARCCOS((−2*(Klig+Kcomp+Lig+Comp−Prot*c)\hat{ }3+9*(Klig+Kcomp+Lig+Comp−Prot*c)*(Kcomp*(Lig−Prot*c)+Klig*(Comp−Prot*c)+Klig*Kcomp)−27*(−1*Klig*Kcomp*Prot*c))/(2*((((Klig+Kcomp+Lig+Comp−Prot*c)\hat{ }2−3*(Kcomp*(Lig−Prot*c)+Klig*(Comp−Prot*c)+Klig*Kcomp))\hat{ }3)\hat{ }0.5)))/3))−(Klig+Kcomp+Lig+Comp−Prot*c)))). “Lig”, “KLig” and “Prot” were all defined values relating to: fluorescent peptide concentration, the Kd of the fluorescent peptide and EphA2 concentration respectively.

Certain bicyclic peptides of the invention were tested in the above mentioned Competition Binding Assay and the results may be seen in Table 1:

TABLE 1
Competition Binding Assay for Selected
Bicyclic Peptides of the Invention

| Bicyclic | | |
|---|---|---|
| Peptide | Mean Kᵢ | Number of |
| Number | (nM) | experiments |
| BCY12854 | 9.339 | 6 |
| BCY12855 | 5.548 | 6 |
| BCY12856 | 4.632 | 8 |
| BCY12857 | 15.99 | 6 |
| BCY12858 | 8.189 | 6 |
| BCY12859 | 0.7232 | 8 |
| BCY12860 | 0.6604 | 6 |
| BCY12861 | 21.74 | 6 |
| BCY12862 | 1.222 | 6 |
| BCY12863 | 5.844 | 6 |
| BCY12864 | 9.295 | 6 |
| BCY12865 | 0.8573 | 6 |
| BCY12866 | 0.244 | 10 |
| BCY13116 | 1.127 | 8 |
| BCY13117 | 1.116 | 8 |
| BCY13118 | 5.425 | 6 |
| BCY13119 | 2.637 | 8 |
| BCY13120 | 0.9158 | 6 |
| BCY13121 | 1.491 | 6 |
| BCY13122 | 7.801 | 8 |
| BCY13123 | 6.441 | 6 |
| BCY13124 | 7.615 | 6 |
| BCY13126 | 25.33 | 6 |
| BCY13127 | 14.9 | 6 |
| BCY13128 | 16.11 | 6 |
| BCY13130 | 7.798 | 6 |
| BCY13131 | 27.08 | 6 |
| BCY13132 | 7.082 | 6 |
| BCY13134 | 1.155 | 6 |
| BCY13135 | 0.8938 | 6 |
| BCY9594 | 1.435 | 38 |
| BCY13133 | 3.237 | 6 |
| BCY13125 | 1.829 | 6 |
| BCY13129 | 1.883 | 6 |
| BCY13917 | 1.715 | 6 |
| BCY13918 | 2.623 | 6 |
| BCY13919 | 2.102 | 6 |
| BCY13920 | 0.6415 | 6 |
| BCY13922 | 1.137 | 6 |
| BCY13923 | 1.322 | 6 |
| BCY14047 | 3.883 | 6 |
| BCY14048 | 4.103 | 6 |

1-9. (canceled)

10. A peptide ligand specific for EphA2, the peptide ligand comprising
(a) a polypeptide comprising at least three reactive groups, separated by at least two loop sequences, and
(b) a molecular scaffold, wherein the molecular scaffold is 1,1′,1″-(1,3,5-triazinane-1,3,5-triyl)triprop-2-en-1-one, and wherein the molecular scaffold forms covalent bonds with the reactive groups of the polypeptide such that at least two polypeptide loops are formed on the molecular scaffold,
wherein the polypeptide comprises an amino acid sequence selected from:
A-[HArg]-D-Cᵢ[HyP][Cba]VNPLCᵢᵢLHP[dD]W[HArg]Cᵢᵢᵢ (SEQ ID NO: 28; herein referred to as BCY12862);
A-[HArg]-D-Cᵢ[HyP][Cba]VNPLCᵢᵢLEP[dD]WTCᵢᵢᵢ (SEQ ID NO: 29; herein referred to as BCY12863);
[dA]-[HArg]-D-Cᵢ[HyP][Cba]VNPLCᵢᵢLEP[dD]WTCᵢᵢᵢ-[dA] (SEQ ID NO: 30; herein referred to as BCY12864);
Cᵢ[HyP][Cba]VNPLCᵢᵢL[3,3-DPA]P[dD]WTCᵢᵢᵢ-[dA] (SEQ ID NO: 31; herein referred to as BCY12865);
[dA]-[HArg]-D-Cᵢ[HyP][Cba]VNPLCᵢᵢLEP[dA]WTCᵢᵢᵢ-[dA] (SEQ ID NO: 37; herein referred to as BCY13123); and
[d1Nal]-[HArg]-D-Cᵢ[HyP][Cba]VNPLCᵢᵢLEP[dA]WTCᵢᵢᵢ-[dA] (SEQ ID NO: 38; herein referred to as BCY13124);
wherein Ac represents acetyl, HyP represents hydroxyproline, HArg represents homoarginine, 3,3-DPA represents 3,3-diphenylalanine, Cba represents β-cyclobutylalanine, 1Nal represents 1-naphthylalanine, and Cᵢ, Cᵢᵢ, and Cᵢᵢᵢ represent first (i), second (ii) and third (iii) cysteine residues;
or a pharmaceutically acceptable salt thereof.

11. The peptide ligand of claim 10, wherein the polypeptide comprises the amino acid sequence
A-[HArg]-D-Cᵢ[HyP][Cba]VNPLCᵢᵢLHP[dD]W[HArg]Cᵢᵢᵢ (SEQ ID NO: 28; herein referred to as BCY12862).

12. The peptide ligand of claim 10, conjugated to a cytotoxic agent, a radiochelator, a metal chelator, or a chromophore.

13. The peptide ligand of claim 10, conjugated to a cytotoxic agent, a radiochelator or a metal chelator.

14. The peptide ligand of claim 10, wherein the peptide ligand comprises an N-terminal effector group.

15. The peptide ligand of claim 14, wherein the N-terminal effector group is a cytotoxic agent.

16. The peptide ligand of claim 14, wherein the N-terminal effector group is a radiochelator or a metal chelator.

17. The peptide ligand of claim 10, wherein the peptide ligand comprises a C-terminal effector group.

18. The peptide ligand of claim 17, wherein the C-terminal effector group is a cytotoxic agent.

19. The peptide ligand of claim 17, wherein the C-terminal effector group is a radiochelator or a metal chelator.

20. The peptide ligand of claim 10, wherein the peptide ligand comprises a C-terminal amide group.

21. The peptide ligand of claim 10, wherein the peptide ligand is in the form of the free acid.

22. The peptide ligand of claim 10, wherein the peptide ligand is a pharmaceutically acceptable salt thereof selected from the group consisting of sodium, potassium, calcium and ammonium.

23. A pharmaceutical composition which comprises the peptide ligand of claim 10, in combination with one or more pharmaceutically acceptable excipients.

24. A method, comprising administering the peptide ligand of claim 10 to a subject.

25. The method of claim 24, wherein the subject has or is suspected of having a disease or disorder characterised by overexpression of EphA2 in diseased tissue.

26. A method of binding EphA2 in an EphA2-expressing cell, the method comprising contacting the cell with the peptide ligand of claim 10.

27. The method of claim 26, wherein the cell is a cancer cell, wherein the cancer is characterised by overexpression of EphA2.


SEQUENCE LISTING (60 sequences)

SEQ ID NO: 1 - protein, 18 aa, synthetic construct
  REGION 1..18: note = Synthetic Peptide
  SITE 2: note = X is homoarginine
  SITE 5: note = X is hydroxyproline
  SITE 17: note = X is homoarginine
  SITE 15: note = D-aspartic acid
AXDCXLVNPLCLHPDWXC

SEQ ID NO: 2 - protein, 19 aa, synthetic construct
  REGION 1..19: note = Synthetic Peptide
  SITE 1: note = X is 4-pentynoic acid
  SITE 3: note = X is homoarginine
  SITE 6: note = X is hydroxyproline
  SITE 18: note = X is homoarginine
  SITE 16: note = D-aspartic acid
XAXDCXLVNPLCLHPDWXC

SEQ ID NO: 3 - protein, 19 aa, synthetic construct
  REGION 1..19: note = Synthetic Peptide
  SITE 2: note = X is homoarginine
  SITE 5: note = X is hydroxyproline
  SITE 17: note = X is homoarginine
  SITE 19: note = X is Lysine conjugated to 4-pentynoic acid
  SITE 15: note = D-aspartic acid
AXDCXLVNPLCLHPDWXCX

SEQ ID NO: 4 - protein, 19 aa, synthetic construct
  REGION 1..19: note = Synthetic Peptide
  SITE 2: note = X is homoarginine
  SITE 5: note = X is hydroxyproline
  SITE 17: note = X is homoarginine
  SITE 15: note = D-aspartic acid
AXDCXLVNPLCLHPDWXCK

SEQ ID NO: 5 - protein, 18 aa, synthetic construct
  REGION 1..18: note = Synthetic Peptide
  SITE 1: note = X is N-methyl-alanine
  SITE 2: note = X is homoarginine
  SITE 5: note = X is hydroxyproline
  SITE 17: note = X is homoarginine
  SITE 15: note = D-aspartic acid
XXDCXLVNPLCLHPDWXC

SEQ ID NO: 6 - protein, 30 aa, synthetic construct
  REGION 1..30: note = Synthetic Peptide
  SITE 1: note = X is 4-pentynoic acid
  SITE 2: note = X is bAla
  REGION 3..12: note = X is sarcosine
  SITE 29: note = X is homoarginine
XXXXXXXXXXXXVGPCLWDPTPCANLHLXC

SEQ ID NO: 7 - protein, 18 aa, synthetic construct
  REGION 1..18: note = Synthetic Peptide
  SITE 2: note = X is homoarginine
  SITE 5: note = X is hydroxyproline
  SITE 13: note = X is Lysine conjugated to 4-pentynoic acid
  SITE 17: note = X is homoarginine
  SITE 15: note = D-aspartic acid
AXDCXLVNPLCLXPDWXC

SEQ ID NO: 8 - protein, 18 aa, synthetic construct
  REGION 1..18: note = Synthetic Peptide
  SITE 2: note = X is homoarginine
  SITE 5: note = X is hydroxyproline
  SITE 6: note = X is Lysine conjugated to 4-pentynoic acid
  SITE 17: note = X is homoarginine
  SITE 15: note = D-aspartic acid
AXDCXXVNPLCLHPDWXC

SEQ ID NO: 9 - protein, 18 aa, synthetic construct
  REGION 1..18: note = Synthetic Peptide
  SITE 2: note = X is homoarginine
  SITE 5: note = X is hydroxyproline
  SITE 12: note = X is Lysine conjugated to 4-pentynoic acid
  SITE 17: note = X is homoarginine
  SITE 15: note = D-aspartic acid
AXDCXLVNPLCXHPDWXC

SEQ ID NO: 10 - protein, 18 aa, synthetic construct
  REGION 1..18: note = Synthetic Peptide
  SITE 2: note = X is homoarginine
  SITE 5: note = X is hydroxyproline
  SITE 17: note = X is homoarginine
  SITE 15: note = D-aspartic acid
AXDCXLVNPLCLKPDWXC

SEQ ID NO: 11 - protein, 18 aa, synthetic construct
  REGION 1..18: note = Synthetic Peptide
  SITE 2: note = X is homoarginine
  SITE 5: note = X is hydroxyproline
  SITE 17: note = X is homoarginine
  SITE 15: note = D-aspartic acid
AXDCXKVNPLCLHPDWXC

SEQ ID NO: 12 - protein, 18 aa, synthetic construct
  REGION 1..18: note = Synthetic Peptide
  SITE 2: note = X is homoarginine
  SITE 5: note = X is hydroxyproline
  SITE 17: note = X is homoarginine
  SITE 15: note = D-aspartic acid
AXDCXLVNPLCKHPDWXC

SEQ ID NO: 13 - protein, 18 aa, synthetic construct
  REGION 1..18: note = Synthetic Peptide
  SITE 2: note = X is homoarginine
  SITE 5: note = X is hydroxyproline
  SITE 17: note = X is homoarginine
  SITE 15: note = D-glutamic acid
AXDCXLVNPLCLHPEWXC

SEQ ID NO: 14 - protein, 18 aa, synthetic construct
  REGION 1..18: note = Synthetic Peptide
  SITE 2: note = X is homoarginine
  SITE 5: note = X is hydroxyproline
  SITE 17: note = X is homoarginine
  SITE 15: note = D-glutamic acid
AXECXLVNPLCLHPEWXC

SEQ ID NO: 15 - protein, 18 aa, synthetic construct
  REGION 1..18: note = Synthetic Peptide
  SITE 2: note = X is homoarginine
  SITE 5: note = X is hydroxyproline
  SITE 17: note = X is homoarginine
  SITE 15: note = D-aspartic acid
AXDCXLVNPLCLEPDWXC

SEQ ID NO: 16 - protein, 18 aa, synthetic construct
  REGION 1..18: note = Synthetic Peptide
  SITE 2: note = X is homoarginine
  SITE 5: note = X is hydroxyproline
  SITE 15: note = D-aspartic acid
AXDCXLVNPLCLHPDWTC

SEQ ID NO: 17 - protein, 18 aa, synthetic construct
  REGION 1..18: note = Synthetic Peptide
  SITE 2: note = X is homoarginine
  SITE 5: note = X is hydroxyproline
  SITE 15: note = D-aspartic acid
AXDCXLVNPLCLEPDWTC

SEQ ID NO: 18 - protein, 19 aa, synthetic construct
  REGION 1..19: note = Synthetic Peptide
  SITE 2: note = X is homoarginine
  SITE 5: note = X is hydroxyproline
  SITE 15: note = D-aspartic acid
AXDCXLVNPLCLEPDWTCA

SEQ ID NO: 19 - protein, 16 aa, synthetic construct
  REGION 1..16: note = Synthetic Peptide
  SITE 2: note = X is hydroxyproline
  SITE 12: note = D-aspartic acid
  SITE 16: note = D-alanine
CXLVNPLCLEPDWTCA

SEQ ID NO: 20 - protein, 18 aa, synthetic construct
  REGION 1..18: note = Synthetic Peptide
  SITE 1: note = X is N-methyl-alanine
  SITE 2: note = X is homoarginine
  SITE 5: note = X is hydroxyproline
  SITE 15: note = D-aspartic acid
XXDCXLVNPLCLEPDWTC

SEQ ID NO: 21 - protein, 18 aa, synthetic construct
  REGION 1..18: note = Synthetic Peptide
  SITE 5: note = X is hydroxyproline
  SITE 15: note = D-aspartic acid
  SITE 1: note = D-alanine
AEDCXLVNPLCLEPDWTC

SEQ ID NO: 22 - protein, 18 aa, synthetic construct
  REGION 1..18: note = Synthetic Peptide
  SITE 5: note = X is hydroxyproline
  SITE 15: note = D-aspartic acid
  SITE 1: note = D-alanine
  SITE 2: note = D-alanine
AADCXLVNPLCLEPDWTC

SEQ ID NO: 23 - protein, 17 aa, synthetic construct
  REGION 1..17: note = Synthetic Peptide
  SITE 4: note = X is hydroxyproline
  SITE 14: note = D-aspartic acid
ADCXLVNPLCLEPDWTC

SEQ ID NO: 24 - protein, 18 aa, synthetic construct
  REGION 1..18: note = Synthetic Peptide
  SITE 2: note = X is homoarginine
  SITE 5: note = X is hydroxyproline
  SITE 15: note = D-alanine
AXDCXLVNPLCLEPAWTC

SEQ ID NO: 25 - protein, 15 aa, synthetic construct
  REGION 1..15: note = Synthetic Peptide
  SITE 2: note = X is hydroxyproline
  SITE 10: note = X is 3,3-diphenylalanine
  SITE 12: note = D-aspartic acid
CXLVNPLCLXPDWTC

SEQ ID NO: 26 - protein, 18 aa, synthetic construct
  REGION 1..18: note = Synthetic Peptide
  SITE 2: note = X is homoarginine
  SITE 5: note = X is hydroxyproline
  SITE 13: note = X is 3,3-diphenylalanine
  SITE 15: note = D-aspartic acid
AXDCXLVNPLCLXPDWTC

SEQ ID NO: 27 - protein, 16 aa, synthetic construct
  REGION 1..16: note = Synthetic Peptide
  SITE 2: note = X is hydroxyproline
  SITE 10: note = X is 3,3-diphenylalanine
  SITE 12: note = D-aspartic acid
CXLVNPLCLXPDWTCK

SEQ ID NO: 28 - protein, 18 aa, synthetic construct
  REGION 1..18: note = Synthetic Peptide
  SITE 2: note = X is homoarginine
  SITE 5: note = X is hydroxyproline
  SITE 6: note = X is Beta-cyclobutylalanine
  SITE 17: note = X is homoarginine
  SITE 15: note = D-aspartic acid
AXDCXXVNPLCLHPDWXC

SEQ ID NO: 29 - protein, 18 aa, synthetic construct
  REGION 1..18: note = Synthetic Peptide
  SITE 2: note = X is homoarginine
  SITE 5: note = X is hydroxyproline
  SITE 6: note = X is Beta-cyclobutylalanine
  SITE 15: note = D-aspartic acid
AXDCXXVNPLCLEPDWTC

SEQ ID NO: 30 - protein, 19 aa, synthetic construct
  REGION 1..19: note = Synthetic Peptide
  SITE 2: note = X is homoarginine
  SITE 5: note = X is hydroxyproline
  SITE 6: note = X is Beta-cyclobutylalanine
  SITE 15: note = D-aspartic acid
  SITE 1: note = D-alanine
  SITE 19: note = D-alanine
AXDCXXVNPLCLEPDWTCA

SEQ ID NO: 31 - protein, 16 aa, synthetic construct
  REGION 1..16: note = Synthetic Peptide
  SITE 2: note = X is hydroxyproline
  SITE 3: note = X is Beta-cyclobutylalanine
  SITE 10: note = X is 3,3-diphenylalanine
  SITE 12: note = D-aspartic acid
CXXVNPLCLXPDWTCA

SEQ ID NO: 32 - protein, 18 aa, synthetic construct
  REGION 1..18: note = Synthetic Peptide
  SITE 2: note = X is homoarginine
  SITE 5: note = X is hydroxyproline
  SITE 13: note = X is 3,3-diphenylalanine
  SITE 17: note = X is homoarginine
  SITE 15: note = D-aspartic acid
AXDCXLVNPLCLXPDWXC

SEQ ID NO: 33 - protein, 18 aa, synthetic construct
  REGION 1..18: note = Synthetic Peptide
  SITE 2: note = X is homoarginine
  SITE 5: note = X is hydroxyproline
  SITE 17: note = X is homoarginine
  SITE 15: note = X is D-1-naphthylalanine
AXDCXLVNPLCLHPXWXC

SEQ ID NO: 34 - protein, 18 aa, synthetic construct
  REGION 1..18: note = Synthetic Peptide
  SITE 2: note = X is homoarginine
  SITE 5: note = X is hydroxyproline
  SITE 13: note = X is 1-naphthylalanine
  SITE 17: note = X is homoarginine
  SITE 15: note = D-aspartic acid
AXDCXLVNPLCLXPDWXC

SEQ ID NO: 35 - protein, 18 aa, synthetic construct
  REGION 1..18: note = Synthetic Peptide
  SITE 2: note = X is homoarginine
  SITE 5: note = X is hydroxyproline
  SITE 15: note = X is D-1-naphthylalanine
AXDCXLVNPLCLEPXWTC

SEQ ID NO: 36 - protein, 15 aa, synthetic construct
  REGION 1..15: note = Synthetic Peptide
  SITE 2: note = X is hydroxyproline
  SITE 10: note = X is 1-naphthylalanine
  SITE 12: note = D-aspartic acid
CXLVNPLCLXPDWTC

SEQ ID NO: 37 - protein, 19 aa, synthetic construct
  REGION 1..19: note = Synthetic Peptide
  SITE 2: note = X is homoarginine
  SITE 5: note = X is hydroxyproline
  SITE 6: note = X is Beta-cyclobutylalanine
  SITE 1: note = D-alanine
  SITE 15: note = D-alanine
AXDCXXVNPLCLEPAWTCA

SEQ ID NO: 38 - protein, 19 aa, synthetic construct
  REGION 1..19: note = Synthetic Peptide
  SITE 1: note = X is D-1-naphthylalanine
  SITE 2: note = X is homoarginine
  SITE 5: note = X is hydroxyproline
  SITE 6: note = X is Beta-cyclobutylalanine
  SITE 15: note = D-alanine
  SITE 19: note = D-alanine
XXDCXXVNPLCLEPAWTCA

SEQ ID NO: 39 - protein, 18 aa, synthetic construct
  REGION 1..18: note = Synthetic Peptide
  SITE 2: note = X is homoarginine
  SITE 5: note = X is hydroxyproline
  SITE 6: note = X is homoglutamic acid
  SITE 17: note = X is homoarginine
  SITE 15: note = D-aspartic acid
AXDCXXVNPLCLHPDWXC

SEQ ID NO: 40 - protein, 18 aa, synthetic construct
  REGION 1..18: note = Synthetic Peptide
  SITE 2: note = X is homoarginine
  SITE 5: note = X is hydroxyproline
  SITE 12: note = X is homoglutamic acid
  SITE 17: note = X is homoarginine
  SITE 15: note = D-aspartic acid
AXDCXLVNPLCXHPDWXC

SEQ ID NO: 41 - protein, 18 aa, synthetic construct
  REGION 1..18: note = Synthetic Peptide
  SITE 2: note = X is homoarginine
  SITE 5: note = X is hydroxyproline
  SITE 13: note = X is homoglutamic acid
  SITE 17: note = X is homoarginine
  SITE 15: note = D-aspartic acid
AXDCXLVNPLCLXPDWXC

SEQ ID NO: 42 - protein, 18 aa, synthetic construct
  REGION 1..18: note = Synthetic Peptide
  SITE 2: note = X is homoarginine
  SITE 5: note = X is hydroxyproline
  SITE 15: note = X is D-norleucine
  SITE 17: note = X is homoarginine
AXDCXLVNPLCLHPXWXC

SEQ ID NO: 43 - protein, 18 aa, synthetic construct
  REGION 1..18: note = Synthetic Peptide
  SITE 2: note = X is homoarginine
  SITE 5: note = X is hydroxyproline
  SITE 13: note = X is norleucine
  SITE 17: note = X is homoarginine
  SITE 15: note = D-aspartic acid
AXDCXLVNPLCLXPDWXC

SEQ ID NO: 44 - protein, 18 aa, synthetic construct
  REGION 1..18: note = Synthetic Peptide
  SITE 2: note = X is homoarginine
  SITE 5: note = X is hydroxyproline
  SITE 17: note = X is homoarginine
  SITE 18: note = X is cysteamine
  SITE 15: note = D-aspartic acid
AXDCXLVNPLCLHPDWXX

SEQ ID NO: 45 - protein, 19 aa, synthetic construct
  REGION 1..19: note = Synthetic Peptide
  SITE 2: note = X is hydroxyproline
  SITE 14: note = X is homoarginine
  SITE 12: note = D-aspartic acid
  SITE 17: note = D-histidine
  SITE 19: note = D-lysine
CXLVNPLCLHPDWXCLHGK

SEQ ID NO: 46 - protein, 16 aa, synthetic construct
  REGION 1..16: note = Synthetic Peptide
  SITE 1: note = X is 3-mercaptopropionic acid
  SITE 2: note = X is hydroxyproline
  SITE 10: note = X is 3,3-diphenylalanine
  SITE 12: note = D-aspartic acid
  SITE 16: note = D-lysine
XXLVNPLCLXPDWTCK

SEQ ID NO: 47 - protein, 18 aa, synthetic construct
  REGION 1..18: note = Synthetic Peptide
  SITE 2: note = X is homoarginine
  SITE 5: note = X is hydroxyproline
  SITE 13: note = X is N3-methyl-L-histidine
  SITE 17: note = X is homoarginine
  SITE 15: note = D-aspartic acid
AXDCXLVNPLCLXPDWXC

SEQ ID NO: 48 - protein, 18 aa, synthetic construct
  REGION 1..18: note = Synthetic Peptide
  SITE 2: note = X is homoarginine
  SITE 5: note = X is hydroxyproline
  SITE 13: note = X is N1-methyl-L-histidine
  SITE 17: note = X is homoarginine
  SITE 15: note = D-aspartic acid
AXDCXLVNPLCLXPDWXC

SEQ ID NO: 49 - protein, 18 aa, synthetic construct
  REGION 1..18: note = Synthetic Peptide
  SITE 2: note = X is homoarginine
  SITE 5: note = X is hydroxyproline
  SITE 13: note = X is beta-(4-thiazolyl)-alanine
  SITE 17: note = X is homoarginine
  SITE 15: note = D-aspartic acid
AXDCXLVNPLCLXPDWXC

SEQ ID NO: 50 - protein, 18 aa, synthetic construct
  REGION 1..18: note = Synthetic Peptide
  SITE 2: note = X is homoarginine
  SITE 5: note = X is hydroxyproline
  SITE 17: note = X is homoarginine
  SITE 15: note = D-aspartic acid
AXDCXLVNPLCLFPDWXC

SEQ ID NO: 51 - protein, 18 aa, synthetic construct
  REGION 1..18: note = Synthetic Peptide
  SITE 2: note = X is homoarginine
  SITE 5: note = X is hydroxyproline
  SITE 13: note = X is 2-thienyl-alanine
  SITE 17: note = X is homoarginine
  SITE 15: note = D-aspartic acid
AXDCXLVNPLCLXPDWXC

SEQ ID NO: 52 - protein, 18 aa, synthetic construct
  REGION 1..18: note = Synthetic Peptide
  SITE 2: note = X is homoarginine
  SITE 5: note = X is hydroxyproline
  SITE 13: note = X is 3-thienylalanine
  SITE 17: note = X is homoarginine
  SITE 15: note = D-aspartic acid
AXDCXLVNPLCLXPDWXC

SEQ ID NO: 53 - protein, 18 aa, synthetic construct
  REGION 1..18: note = Synthetic Peptide
  SITE 2: note = X is homoarginine
  SITE 5: note = X is hydroxyproline
  SITE 17: note = X is homoarginine
  SITE 15: note = D-aspartic acid
AXDCXLVNPLCLNPDWXC

SEQ ID NO: 54 - protein, 18 aa, synthetic construct
  REGION 1..18: note = Synthetic Peptide
  SITE 2: note = X is homoarginine
  SITE 5: note = X is hydroxyproline
  SITE 17: note = X is homoarginine
  SITE 15: note = D-aspartic acid
AXDCXLVNPLCLQPDWXC

SEQ ID NO: 55 - protein, 18 aa, synthetic construct
  REGION 1..18: note = Synthetic Peptide
  SITE 2: note = X is homoarginine
  SITE 5: note = X is hydroxyproline
  SITE 13: note = X is Lysine conjugated to 4-pentynoic acid
  SITE 17: note = X is homoarginine
  SITE 15: note = D-aspartic acid
AXDCXLVNPLCLXPDWXC

SEQ ID NO: 56 - protein, 19 aa, synthetic construct
  REGION 1..19: note = Synthetic Peptide
  SITE 1: note = X is 4-pentynoic acid
  SITE 3: note = X is homoarginine
  SITE 6: note = X is hydroxyproline
  SITE 18: note = X is homoarginine
  SITE 16: note = D-aspartic acid
XAXDCXLVNPLCLKPDWXC

SEQ ID NO: 57 - protein, 18 aa, synthetic construct
  REGION 1..18: note = Synthetic Peptide
  SITE 2: note = X is homoarginine
  SITE 5: note = X is hydroxyproline
  SITE 13: note = X is N-(4-pentynoyl)-L-lysine-(N2-((S)-4-carboxy-4- palmitamidobutanoyl)-N6-diazo-L-lysine)
  SITE 17: note = X is homoarginine
  SITE 15: note = D-aspartic acid
AXDCXLVNPLCLXPDWXC

SEQ ID NO: 58 - protein, 19 aa, synthetic construct
  REGION 1..19: note = Synthetic Peptide
  SITE 1: note = X is N2-((S)-4-carboxy-4-palmitamidobutanoyl)-N6-di azo-L-lysine[4-pentynoic acid]
  SITE 3: note = X is homoarginine
  SITE 6: note = X is hydroxyproline
  SITE 18: note = X is homoarginine
  SITE 16: note = D-aspartic acid
XAXDCXLVNPLCLKPDWXC

SEQ ID NO: 59 - protein, 18 aa, synthetic construct
  REGION 1..18: note = Synthetic Peptide
  SITE 2: note = X is homoarginine
  SITE 5: note = X is hydroxyproline
  SITE 13: note = X is para-carboxy-phenylalanine
  SITE 17: note = X is homoarginine
  SITE 15: note = D-aspartic acid
AXDCXLVNPLCLXPDWXC

SEQ ID NO: 60 - protein, 27 aa, synthetic construct
  REGION 1..27: note = Synthetic Peptide
  SITE 1: note = X is 5/6-carboxfluorescein
  REGION 3..7: note = X is sarcosine
XGXXXXXADVTCPWGPFWCPVNRPGCA